(19) United States
(12) Patent Application Publication
(10) Pub. No.: US 2026/0240520 A1
(43) Pub. Date: Aug. 20, 2026

(54) GARMENT WITH ULTRASONIC IMAGING SENSORS

(71) Applicant: IKKO Health Ltd., Tel Aviv (IL)

(72) Inventors: Ramon AXELROD, Kiryat Ono (IL); Pinchas CHAVIV, Hod HaSharon (IL); Michal COHEN SFADY, Ramat Gan (IL)

(73) Assignee: IKKO Health Ltd., Tel Aviv (IL)

(21) Appl. No.: 19/649,359

(22) Filed: Apr. 16, 2026

Related U.S. Application Data
(63) Continuation of application No. 18/997,806, filed on Aug. 1, 2023.
(60) Provisional application No. 63/394,362, filed on Aug. 2, 2022.

(51) Int. Cl. A61B 8/00; G01S 7/52; G01S 15/89

(52) U.S. Cl. CPC A61B 8/429; A61B 8/4236; A61B 8/4281; A61B 8/4494; A61B 8/462; A61B 8/52; G01S 7/52004; G01S 7/52076; G01S 15/8929; G01S 15/8952

(57) ABSTRACT

A garment has ultrasonic sensors placed on a patient's body, which solves problems associated with poor contact between the ultrasonic sensors and the portion to be imaged, by using feedback to improve contact and/or to eliminate transceivers with bad emission patterns.

### CROSS-REFERENCE TO RELATED APPLICATIONS

This application is a continuation of U.S. patent application Ser. No. 18/997,806 filed Jan. 23, 2025, which is a National Phase Application of PCT International Application No. PCT/IB 2023/057803 filed Aug. 1, 2023, which claims priority to U.S. Provisional Application No. 63/394,362 filed Aug. 2, 2022, all of which are hereby incorporated by reference in their entirety.

### FIELD OF THE INVENTION

The present invention generally relates to ultrasonic imaging, and particularly to a garment that has ultrasonic sensors placed on a patient's body, which can acquire ultrasonic images of body parts without using gel, water or any other non-solid coupling medium.

### BACKGROUND OF THE INVENTION

Ultrasound (US) is one of the most widely available medical imaging modalities. Ultrasound is mainly used to image soft tissues and is considered safe. Current ultrasound systems consist of an ultrasonic pod or transducer that emits and receives ultrasound signals, and accompanying hardware that analyzes the received or reflected signals and generates an image. Medical ultrasound typically operates at frequencies between 3 MHz and 15 MHz, although 1 MHz also exists in older type equipment.

One critical issue is maintaining excellent ultrasonic contact between the US pod and the patient's skin. Excellent contact implies, in more scientific terms, that most of the energy output from the US pod is delivered to the patient's body with negligible delay. In engineering terms excellent ultrasonic contact means no air between the US pod and the body and good impedance matching between the US pod and the patient's body.

Standard US transducers are built with an array that contains a large number of small US emitters/receivers. These emitters are operated in a very specific mode so as to emit a focused beam into the patient's body (like phased arrays). In order to generate the focused beam, US systems use precise timing when operating the individual emitters within an US pod. US systems perform well if the ultrasonic contact between the pod and the patient is excellent. However, if the US array in the pod is not in sufficiently good ultrasonic contact with the patient's body, then focused beams will not form and image acquisition will not be possible, or significantly degrade. Achieving excellent ultrasonic contact over the entire area of an US pod, approximately 70 mm×25 mm or larger, can be very difficult without a coupling medium.

Standard US solves the “excellent ultrasonic contact” problem by using a matching layer deposited on the US pod body facing surface, and by using a gel to remove air from the gaps between the US pod and the patient's body/skin. This is the reason why in every US procedure the patient's body is covered with a large amount of gel. The gel ensures that even if the patient's physique does not enable the entire US pod to be in direct contact with the patient's body, the gel will fill the voids between pod and body and will ensure proper ultrasonic contact.

Over the last decade there has been significant interest in a new form of ultrasonic imaging modality. This new modality attempts to use multiple ultrasonic transceivers that are located on large areas on the body and, in some forms, around the body. The ultrasonic transceivers in this new mode are much smaller than standard US pods-less than 10×10 mm and cover a much larger area of the body when compared to classic medical US. Unlike classical US, which uses focused beams, reflections and time-of-flight parameters to generate an image, the new modality uses reflections, transmission and refractions to extract more physical data and generate much clearer, 3D, images.

However, the problem of guaranteeing excellent ultrasonic contact remains. Without excellent ultrasonic contact the new modality cannot generate proper images. Using gel or any other liquid material, in the case of the new imaging modality, is highly undesirable since, unlike classical US, it requires to cover an entire body part and not just a limited area.

### SUMMARY OF THE INVENTION

The present invention seeks to provide a garment that has ultrasonic sensors placed on a patient's body, which can acquire ultrasonic images of body parts and which solves problems associated with poor contact between the ultrasonic sensors and the portion to be imaged, by using feedback to improve contact and/or to eliminate transceivers with bad emission patterns, as is described more in detail hereinbelow.

One embodiment includes a large number of transceivers placed on a patient's body individually, in large patches or embedded in a garment. In the case of a garment it is elastic and attempts to adhere to the shape of the patient's body. The garment can take the form of a belt, hat, sleeve, vest, shirt, pants or any combination of those.

Unlike classical US, which images single organs, the present invention provides a novel imaging modality for full body part imaging (e.g., abdomen, pelvis, limb, neck), not for a specific organ within a body part.

### BRIEF DESCRIPTION OF THE DRAWINGS

The present invention will be understood and appreciated more fully from the following detailed description, taken in conjunction with the drawings in which:

FIG. 1A is an illustration of a prior art ultrasonic emitter assembly and an ultrasonic sensor assembly on one side of a tested specimen.

FIG. 1B is an illustration of a prior art ultrasonic emitter assembly, wherein if air is present between the emitter and the surface of the tested specimen (skin), a large portion of the signal will be reflected much faster, along with 180° phase inversion.

FIGS. 2A-2D are simplified illustrations of examples of frequency dependent transmission (frequency of 2A>2B>2C>2D), in accordance with a non-limiting embodiment of the invention;

FIGS. 3A-3D are simplified illustrations of examples of frequency dependent transmission (frequency of 2A>2B>2C>2D), in accordance with a non-limiting embodiment of the invention, but with an air pocket found in the body;

FIG. 4A is a simplified illustration of an emission pattern from the emitter with no small attenuator present;

FIG. 4B is a simplified illustration of emission patterns from the emitter with a small attenuator present in the body; and

FIG. 5 is a simplified pictorial illustration of measurements of the reflected signal (or surface waves) taken using at least two portions A and B of adjacent emitters of the same transceiver.

### DETAILED DESCRIPTION OF EMBODIMENTS

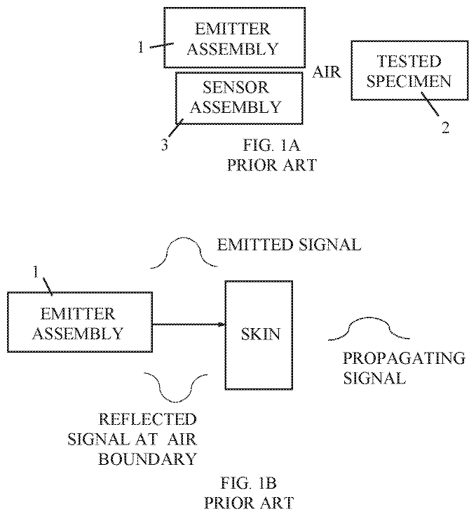

Reference is made to FIG. 1A, which illustrates an ultrasonic emitter assembly 1 and an ultrasonic sensor assembly 3 on one side of a tested specimen 2. Local reflection occurs when an ultrasonic blocking material (e.g. air) completely separates the entire, or most of, the emitter's area from the patient body.

In this mode a high frequency (e.g., 5 MHz) pulse is emitted and the immediate/short-term reflection is measured at the emitter location. In the case of a good ultrasonic contact, the signal will propagate into the tested specimen and reflections will occur at surfaces lying at least 5 mm from the emitter and be very low. In this case, the first returned signal will arrive several microseconds after the test pulse is emitted, with an intensity that is several percent of the emitted signal.

As seen in FIG. 1B, if air is present between the emitter and the surface of the tested specimen (skin), a large portion of the signal will be reflected much faster, along with 180° phase inversion.

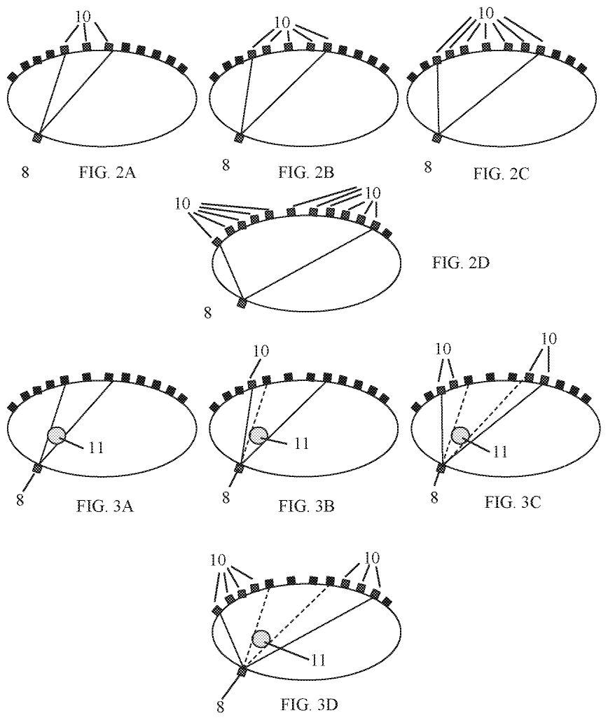

Reference is made to FIGS. 2A-2D, which illustrate examples of frequency dependent transmission, used in an embodiment of the present invention.

Frequency dependent transmission occurs when an ultrasonic blocking material (e.g., air) volume is sufficient to allow low frequency signals to penetrate the patient's body while reflecting the high frequencies locally.

In this mode, the system uses attenuation information gathered from multiple sensors that are not located at the emitter location, at multiple frequencies to estimate the quality of ultrasonic contact.

If the emitter has a very narrow radiation pattern and the emitted waveform passes through soft tissues, there will be sensors that will be able to receive the signal. However, if the pressure wave encounters an attenuating layer such as air (e.g., lungs or intestine) the signal will be severely attenuated and no sensor will be able to detect it.

However, if the same emitter operates at a lower frequency, then when a lower frequency wave is sent through the same aperture its radiation pattern is wider. As the operating frequency is decreased, the radiation pattern, or more specifically the emission angle, becomes wider and, for the same emitter, more sensors will be able to receive the emitted signal.

FIGS. 2A-2D illustrate an emitter 8 which emits ultrasonic signals at successively decreasing operating frequencies F1 to F4, wherein F1>F2>F3>F4. It is seen that as the frequency decreases, more sensors 10 are able to receive the emitted signal.

FIGS. 3A-3D illustrate emitter 8 emitting the same ultrasonic signals at the same successively decreasing operating frequencies F1 to F4, but with an air pocket 11 found in the body. As the radiation angle increases (for decreasing frequency), more sensors 10 are able to sense the transmitted signal.

If the group of sensors that receive a signal when F1 is used is designated Gf1, and the group of sensors that receive a signal when F2 is used Gf2, etc., then if there is good ultrasonic contact at the emitter point, one can assume Gf1<=Gf2<=Gf3<Gf4.

Gf1 may be 0 if the narrow beam encounters a high attenuation area (like a lung). However, as the frequency decreases, the emission angle increases so there will be sensors that receive a signal.

Case #1: Gf1 or {Gf1 and Gf2} are 0 and Gf 3 and Gf 4 are non-zero. This may be a case of weak ultrasonic contact and a local “push” can strengthen the local ultrasonic contact.

Case #2: Gf1 or {Gf1 and Gf2″ or {Gf1 and Gf2 and Gf 3} are 0 and Gf 4 is non-zero. This may be a case of a very weak ultrasonic contact where a local push may help.

Case #3: All Gfis are 0. This may mean either a bad transmitter or a local blocking bone.

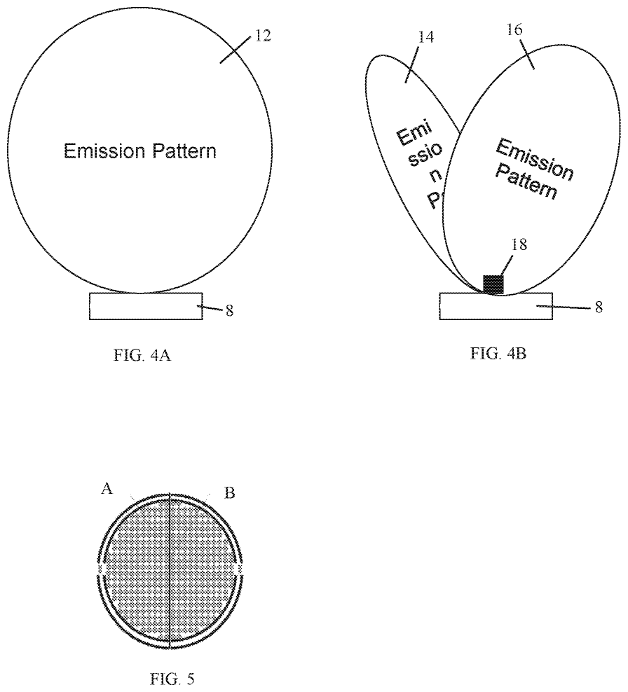

The problem of a local small attenuator in the body is now discussed with reference to FIGS. 4A and 4B. FIG. 4A illustrates an emission pattern 12 from emitter 8 with no small attenuator present. FIG. 4B illustrates emission patterns 14 and 16 from emitter 8 with a small attenuator 18 present in the body.

A local small attenuator is an attenuator that interferes with the emitted wave within the near-field range, thereby distorting its spatial pattern.

When an inversion process takes place the software uses an inverted model of the emitters to simulate the propagating wave. If, however, an attenuator causes a major change in the emission pattern (as in FIG. 4B) then the effect on the inversion process can be severe.

Small attenuators can take the form of a small air bubble that causes some of the signal to pass and be reflected from deeper layers and some to be reflected from the bubble itself. This can lead to changes in the emission pattern rather than a large loss of signal. To avoid this, as seen in FIG. 5, measurements of the reflected signal (or surface waves) are taken using at least two portions A and B of adjacent emitters of the same transceiver. The system first transmits from portion A and analyzes the signals received in portion B, and then vice versa. If there is a small air bubble present, the reflection patterns received by portions A and B are expected to be significantly different. In case of a significant difference between the reflection patterns of two different areas within the transceiver, the specific transceiver will be omitted from the image processing.

The present invention overcomes the ultrasonic contact problem without using a coupling agent. For example, in one embodiment, multiple ultrasonic frequencies are used to determine the quality of ultrasonic contact. Starting from the highest operational frequency, e.g., 1-2 MHz, the system performs “contact testing” by emitting a pulse from each transceiver, recording the received signal at all other transceivers and counting the number of transceivers that received a signal. After testing at the highest frequency, the test is repeated at lower frequencies with the lowest frequency at the range of 20-300 KHz.

A transceiver that fails to communicate with other transceivers in all the tested frequencies (all Gfi are 0) is declared “bad”.

A transceiver that communicates with other transceivers at the highest frequency is declared “excellent”.

Transceivers that test good in several lower frequencies and bad in the higher frequencies are flagged for “manual intervention” to improve the ultrasonic contact.

If after manual intervention a transceiver is still exhibiting ultrasonic contact at few lower frequencies and no ultrasonic contact at high frequencies, it is flagged as “problematic” to the analysis software.

The selection process is based on the physical fact that air attenuation is very high (~100-150 dB/m) for a 1 MHz signal but is fairly low (~0.5 dB/m) in the 30-80 KHz range, allowing the signal to get through small air gaps to the skin.

It is noted that physically there is also an issue with reflections which is ignored at this time.

In one embodiment, “bad” transceivers may be eliminated from the scanning/imaging process.

In one embodiment, unlike standard US systems, unfocused sources are used to scan a body part. There is no need for any timing relation between different transceivers; they can be operated sequentially, in parallel, in groups, etc.

As part of the scanning process transceivers that do not exhibit ultrasonic contact in both the highest and lowest frequencies are declared “bad” transceivers and are flagged so the imaging system does not use them for the scan and imaging process.

In addition, transceivers that show significant differences in the reflection patterns received from two adjacent parts of the same transceiver, are eliminated from the scan and imaging process.

This is not done in prior art US systems. In the prior art, all the elements (pixels) in an US array are used to generate a focused beam.

In one embodiment, feedback may be provided to the patient or medical professional as to the location of transceivers or areas that are “almost good” ultrasonic contact, and guide them to enhance the quality of the contact.

If a transceiver is declared “bad” on the maximum frequency and “good” at the lowest frequency, a signal may be sent to the patient or medical professional to tap or press the relevant area and re-run the contact testing

1. An ultrasonic system comprising:
a plurality of ultrasonic transceivers configured to emit and receive ultrasonic signals when in contact with a body of a subject, wherein the ultrasonic system is configured to:
emit, by a selected ultrasonic transceiver of the plurality of ultrasonic transceivers, a first ultrasonic signal at a first frequency;
determine a first group of ultrasonic transceivers of the plurality of ultrasonic transceivers other than the selected ultrasonic transceiver that receive the first ultrasonic signal;
emit, by the selected ultrasonic transceiver, a second ultrasonic signal at a second frequency lower than the first frequency;
determine a second group of ultrasonic transceivers of the plurality of ultrasonic transceivers other than the selected ultrasonic transceiver that receive the second ultrasonic signal;
determine whether or not the selected ultrasonic transceiver exhibits sufficient contact with the body based on a comparison of a number of ultrasonic transceivers in the first group and the second group; and
upon determination that the selected ultrasonic transceiver does not exhibit sufficient contact with the body, guiding a user to adjust adherence of the selected ultrasonic transceiver to improve contact with the body.

2. The ultrasonic system of claim 1, wherein the plurality of ultrasonic transceivers are configured to contact the body without a coupling medium.

3. The ultrasonic system of claim 1, wherein the first frequency of the first ultrasonic signal is in a megahertz range, and the second frequency of the second ultrasonic signal is in a kilohertz range.

4. The ultrasonic system of claim 1, wherein the first frequency is 1 MHz or higher and the second frequency is within a range of 20-300 KHz.

5. The ultrasonic system of claim 1, wherein the ultrasonic system is configured to determine that the selected ultrasonic transceiver exhibits sufficient contact upon determination that the first group includes at least one ultrasonic transceiver.

6. The ultrasonic system of claim 1, wherein the ultrasonic system is configured to determine that the selected ultrasonic transceiver exhibits sufficient contact upon determination that the second group includes more ultrasonic transceivers than the first group.

7. The ultrasonic system of claim 1, wherein the ultrasonic system is configured to determine that the selected ultrasonic transceiver exhibits insufficient contact upon determination that the first group includes no ultrasonic transceivers and the second group includes at least one ultrasonic transceiver.

8. The ultrasonic system of claim 1, wherein upon determination that both the first group and the second group include no ultrasonic transceivers, the ultrasonic system is configured to exclude the selected ultrasonic transceiver from an imaging process.

9. The ultrasonic system of claim 1, wherein the ultrasonic system is configured to determine that the selected ultrasonic transceiver exhibits insufficient contact upon detection, by the selected ultrasonic transceiver, of a reflection of the transmitted first ultrasonic signal with a phase inversion relative to the transmitted first ultrasonic signal.

10. The ultrasonic system of claim 1, wherein the ultrasonic system is configured to exclude the selected ultrasonic transceiver that exhibits insufficient contact from an ultrasonic imaging process, an ultrasonic inversion process, or both.

11. The ultrasonic system of claim 1, wherein the ultrasonic system is configured to:
emit, by a first portion of emitters of the selected ultrasonic transceiver, a third ultrasonic signal;
receive, by a second portion of the emitters of the selected ultrasonic transceiver, a first reflection of the third ultrasonic signal;
emit, by the second portion of the emitters of the selected ultrasonic transceiver, the third ultrasonic signal;
receive, by the first portion of the emitters of the selected ultrasonic transceiver, a second reflection of the third ultrasonic signal; and
exclude the selected ultrasonic transceiver from an ultrasonic imaging process, an ultrasonic inversion process, or both upon determination that a difference between the first reflection and the second reflection exceeds a difference measure.

12. The ultrasonic system of claim 1, wherein the ultrasonic system is configured to transmit an indication identifying the selected ultrasonic transceiver exhibits insufficient contact for guiding the adjustment of a contact of the selected ultrasonic transceiver to the body.

13. The ultrasonic system of claim 1, wherein the ultrasonic system is configured to perform the contact evaluation for each ultrasonic transceiver of the plurality of ultrasonic transceivers.

14. An ultrasonic system comprising:
a plurality of ultrasonic transceivers configured to emit and receive ultrasonic signals when in a contact with a body of a subject;
wherein the ultrasonic system is configured to:
emit, by a selected ultrasonic transceiver of the plurality of ultrasonic transceivers, a plurality of ultrasonic signals one after another, wherein each ultrasonic signal of the plurality of ultrasonic signals has a frequency lower than a frequency of a preceding ultrasonic signal of the plurality of ultrasonic signals;
for each ultrasonic signal of the plurality of ultrasonic signals, determine a group of ultrasonic transceivers other than the selected ultrasonic transceiver that receive the respective ultrasonic signal to provide a plurality of groups of ultrasonic transceivers;
determine whether or not the selected ultrasonic transceiver exhibits sufficient contact with the body based on a comparison of a number of ultrasonic transceivers in two or more groups of the plurality of groups; and
upon determination that the selected ultrasonic transceiver does not exhibit sufficient contact with the body, guiding a user to adjust adherence of the selected ultrasonic transceiver to improve contact with the body.

15. The ultrasonic system of claim 14, wherein the plurality of ultrasonic transceivers are configured to contact the body without a coupling medium.

16. The ultrasonic system of claim 14, wherein a highest frequency of the plurality of ultrasonic signals is 1 MHz or higher and a lower frequency of the plurality of ultrasonic signals is within a range of 20-300 KHz.

17. The ultrasonic system of claim 14, wherein the ultrasonic system is configured to determine that the selected ultrasonic transceiver exhibits sufficient contact with the body upon determination that a group of the plurality of groups corresponding to reception of an ultrasonic signal of the plurality of ultrasonic signals having a highest frequency includes at least one ultrasonic transceiver.

18. The ultrasonic system of claim 14, wherein the ultrasonic system is configured to determine that the selected ultrasonic transceiver exhibits sufficient contact with the body upon determination that, for successive ultrasonic signals of the plurality of ultrasonic signals having successively lower frequencies, a number of ultrasonic transceivers in a corresponding group of the plurality of groups is equal to or greater than a number of ultrasonic transceivers in a group corresponding to a preceding ultrasonic signal.

19. The ultrasonic system of claim 14, wherein the ultrasonic system is configured to determine that the selected ultrasonic transceiver exhibits insufficient contact with the body upon determination that for one or more ultrasonic signals of the plurality of ultrasonic signals having higher frequencies, each of corresponding one or more groups of the plurality of groups includes no ultrasonic transceivers, and for one or more ultrasonic signal of the plurality of ultrasonic signals having lower frequencies, each of corresponding one or more groups of the plurality of groups includes at least one ultrasonic transceiver.

20. The ultrasonic system of claim 14, wherein upon determination that all groups of the plurality of groups include no ultrasonic transceivers, the ultrasonic system is configured to eliminate the selected ultrasonic transceiver from an imaging process.
